(19) United States
(12) Patent Application Publication
(10) Pub. No.: US 2006/0057591 A1
(43) Pub. Date: Mar. 16, 2006

(54) Method for preventing viral infection

(75) Inventors: Edward L.G. Pryzdial, Vancouver (CA); Melanie C. Derry, Montreal (CA)

Correspondence Address: OGILVY RENAULT LLP, 1981 MCGILL COLLEGE AVENUE, SUITE 1600, MONTREAL, QC, H3A2Y3, CA

(73) Assignee: Canadian Blood Services, Ottawa (CA)

(21) Appl. No.: 10/940,642

(22) Filed: Sep. 15, 2004

(51) Int. Cl. C12Q 1/70; C12Q 1/68

(52) U.S. Cl. 435/6; 435/5

(57) ABSTRACT

There is provided a method for preventing or treating viral infections using members of the annexin proteins family. In particular annexins 1 and 5 as well as inhibitors of heterotetrameric annexin 2, such as antibodies, significantly reduce the production of virus progeny.

### CROSS-REFERENCE TO RELATED APPLICATIONS

This is the first application filed for the present invention.

### TECHNICAL FIELD

This application relates to the prevention of viral infections and more specifically the prevention of viral infections by modulating the functions and/or interactions of annexins or analogs thereof.

### BACKGROUND OF THE INVENTION

Viral infection remains an important health problem in both humans and animals with adverse economic and social consequences. Many antivirals have been developed that are specifically aimed at inhibiting viral genes products implicated, for example, in the viral replication machinery. While these types of antivirals are useful, problems, such as bioavailability, have limited their efficacy.

The mechanisms of entry of viral particle into cells have been extensively studied in the hope of providing targets for therapeutic applications. Annexins, for example, have been implicated in membrane fusion events and it has been suggested that annexin 2 (AnxA2) may be involved in virus entry into cells although this hypothesis has been refuted (Pietropaolo, R. L., Compton, 1999, T. J Gen Virol; 80 (Pt 7):1807-1816).

AnxA2 belongs to the widely expressed annexin protein family, functionally characterized by Ca²⁺ dependent anionic phospholipid binding. AnxA2 exists in at least two configurations: a 36-kDa monomer (p36), the structure typical of annexins, and a heterotetramer (AnxA2t), which consists of two p36 subunits non-covalently bridged by a dimer of p11 subunits (Glenney, Jr. et al., 1986, J. Biol. Chem. 261:10485-10488). The interaction between p36 and p11, an S100 protein family member (S100A10), is Ca²⁺-independent and is mediated by the unique N-terminus of p36 (Becker et al., 1990; Glenney, Jr. et al., 1985; Johnsson et al., 1988). While p36 requires millimolar Ca²⁺ concentrations to bind anionic phospholipid, AnxA2t has a much lower Ca²⁺ requirement in the micromolar range (Evans, Jr. et al., 1994). Both p36 and AnxA2t can aggregate anionic phospholipid-containing membranes (Blackwood et al., 1990; Liu et al., 1997), and AnxA2t has also been implicated in membrane fusion (Chattopadhyay et al., 2003; Harder et al., 1993; Konig et al., 1998). Although the in vivo role of AnxA2 is unknown, its in vitro aggregative and fusogenic properties suggest a role in intracellular trafficking and secretion (Ali et al., 1989; Chasserot-Golaz et al., 1996; Drust et al., 1988; Liu et al., 1997).

In view of the above it is desirable to further our understanding of the role of annexins in viral infection and to provide new targets for inhibiting viral replication.

### SUMMARY OF THE INVENTION

There is therefore provided a method for inhibiting viral infections in animals comprising modulating the functions of annexins. The annexins advantageously provide novel targets and effectors for modulating viral infection.

Thus in one aspect of the present invention there is provided a method to inhibit viral infection (and propagation) comprising inhibiting Annexin 2 (AnxA2). To that effect, antibodies to tetrameric AnxA2 can be used. In a preferred embodiment pqlyclonal anti-p11 antibody against p11 residues 21-38 and polyclonal anti-p36 antibody against p36 residues 9-30 are used. However it will be appreciated that the person skilled in the art, using the guidance of the examples provided below, would be able to identify other antibodies directed to AnxA2 and capable of inhibiting viral infection.

Cells susceptible to viral infection may thus be contacted with anti-p11 and/or anti-p36 antibodies, as described below, at a concentration and for a time sufficient to inhibit or reduce viral infection.

The inventors have shown that annexin 1 (AnxA1) and 5 (AnxA5) can inhibit viral infection. Thus, in another aspect of the invention, there is also provided a method to inhibit viral infection (and propagation) comprising contacting cells susceptible to viral infection with AnxA1 and/or AnxA5 at a concentration and for a time sufficient to inhibit or reduce viral infection.

Anti-p11, anti-p36, AnxA1, AnxA5 or a combination thereof can be administered to an animal in need thereof using method well known in the art. In addition, the above mentioned compounds can be used in combination with one or more known anti-viral agents to enhance the efficacy of the treatment. Anti-viral agents may comprise, but are not limited to, acyclovir, viroptic, idoxirudine, saquinavir, nevirapine and the like.

In particular the above mentioned compounds may be part of a pharmaceutical composition that can be administered intravenously, intramuscularly, subcutaneously, intraperitoneously or intraarterially or a combination thereof. The pharmaceutical composition may also comprise a pharmaceutically acceptable carrier. The administration of the pharmaceutical composition may also be topical to treat or prevent viral infections on the skin or mucosal surfaces for example. Pharmaceutically acceptable carrier for topical composition are well known in the art.

Viral infection mediated by AnxA2 may also be inhibited or reduced by preventing the transcription and/or the translation of the gene coding for AnxA2. For example, anti-sense RNA therapy techniques are envisioned to be covered by the present invention.

The method of the present invention for preventing or reducing viral infection can be applied to any virus the infection cycle of which is dependent in part on AnxA2. Preferably, the method can be applied to, without being limited to, cell membrane-enveloped viruses such as the cytomegalovirus (CMV).

In yet another aspect of the invention, there is provided a method for assessing the susceptibility of an animal to viral infection comprising measuring the levels of AnxA1, AnxA2 and/or AnxA5 in a given tissue of an animal. Increased levels of AnxA2 indicating an increase susceptibility to viral infection and increased levels of AnxA1 and/or AnxA5 indicating a decrease susceptibility to viral infection. The relative levels of AnxA2 vs. AnxA1 and/or AnxA5 may also be indicative of susceptibility to viral infection. More specifically, increased ratios of AnxA2 vs AnxA1 and/or AnxA5 indicating a higher susceptibility and a decrease in that ratio indicating a lower susceptibility.

It is also possible to take advantage of the properties of AnxA1, AnxA2 and AnxA5 with regard to viral infection for increasing the efficiency of viral entry into cells for the purpose of therapy such as gene therapy mediated by viral vectors. Thus, for example, exogenous AnxA2 can be added to cells targeted for therapy thereby increasing viral entry into such cells. A similar effect can be achieved by reducing the inhibitory effect of AnxA1 and 5.

Modulation of viral infection efficiency can also be achieved by modifying the relative amount of annexin types at the surface of the cells using therapeutics such as annexin analogs.

### BRIEF DESCRIPTION OF THE DRAWINGS

Further features and advantages of the present invention will become apparent from the following detailed description, taken in combination with the appended drawings, in which:

FIG. 1A is a graphic showing the effect of endogenous annexin 2 on productive CMV infection in HFF in which HFF were inoculated with CMV at an MOI of 0.0003 in the presence of the indicated concentrations of anti-p11 (∘) or anti-p36(●) antibody; on day 10 post-infection, supernatants were collected and used to inoculate fresh HFF and at 20 hours post-infection, cell lysates were collected and subject to anti-IE72 Western blot analysis; error bars represent standard error; Rsqr=0.99 (anti-p11), 0.97 (antip36); p<0.01;

FIG. 1B represents a bar graph of specificity controls for immuno-inhibition experiments which were conducted as follows: no antibody (a), 100 nM anti-p11 (b), 100 nM anti-p11+100 nM AnxA2t (c), 100 nM anti-p36 (d), 100 nM anti-p36+100 nM AnxA2t (e), 100 nM non-immune rabbit IgG (f) and where each point represents the average change in IE72 expression from two separate wells; error bars represent standard error; Rsqr=0.99 (anti-p11), 0.97 (antip36); p<0.01;

FIG. 1C is a graphic showing the effect of endogenous annexin 2 on productive CMV infection in HFF in which HFF were inoculated with CMV at an MOI of 0.0003 in the presence of the indicated concentrations of anti-p11 (∘) or anti-p36 (●) antibody; on day 10 post-infection, supernatants were collected and used to inoculate fresh HFF and plaques were quantified on day 7 post-infection; error bars represent standard error; Rsqr=0.98 (anti-p11), 0.98 (anti-p36); p<0.01;

FIG. 1D represents a bar graph of specificity controls for immuno-inhibition experiments which were conducted as follows: no antibody (a), 100 nM anti-p11 (b), 100 nM anti-p11+100 nM AnxA2t (c), 100 nM anti-p36 (d), 100 nM anti-p36+100 nM AnxA2t (e), 100 nM non-immune rabbit IgG (f) and where each point represents the average change in plaque number from two separate wells; error bars represent standard error; Rsqr=0.98 (anti-p11), 0.98 (anti-p36); p<0.01;

FIG. 2A is a graphic showing the effect of endogenous annexin 2 on early CMV infection stages in HFF in which HFF were inoculated with CMV at an MOI of 0.003 in the presence of the indicated concentrations of anti-p11 (∘) or anti-p36 (●) antibody and at 20 hours post-infection, lysates were collected and subject to anti-IE72 Western blot analysis; error bars represent standard deviation; Rsqr=0.99 (anti-p11), 0.95 (anti-p36); p<0.05;

FIG. 2B represents a bar graph of specificity controls for immuno-inhibition experiments were conducted as follows: no antibody (a), 100 nM anti-p11 (b), 100 nM anti-p11+100 nM AnxA2t (c), 100 nM anti-p36 (d), 100 nM anti-p36+100 nM AnxA2t (e), and 100 nM non-immune rabbit IgG (f); each point represents the average change in IE72 expression from two separate experiments, comprising two wells each and error bars represent standard deviation; Rsqr=0.99 (anti-p11), 0.95 (anti-p36); p<0.05;

FIG. 2C is a graphic showing the effect of endogenous annexin 2 on early CMV infection stages in HFF in which HFF were inoculated with CMV at an MOI of 0.0003 in the presence of the indicated concentrations of anti-p11 (∘) or antip36 (●) antibody, plaques were quantified on day 7 post-infection, error bars represent standard deviation; Rsqr=0.98 (anti-p11), 0.98 (anti-p36); p<0.05;

FIG. 2D represents a bar graph of specificity controls for immuno-inhibition experiments (inset) were conducted as follows: no antibody (a), 100 nM anti-p11 (b), 100 nM anti-p11+100 nM AnxA2t (c), 100 nM anti-p36 (d), 100 nM anti-p36+100 nM AnxA2t (e), 100 nM non-immune rabbit IgG (f); each point represents the average change in plaque number from four separate experiments, comprising two wells each, error bars represent standard deviation; Rsqr=0.98 (anti-p11), 0.98 (anti-p36); p<0.05;

FIG. 3A is a graphic showing the effect of purified annexin 2 on productive CMV infection in HFF in which HFF were inoculated with CMV at an MOI of 0.0003 in the presence of the indicated concentrations of purified AnxA2t (∘), p11 (●), or p36 ( [symbol] ), on day 10 post-infection, supernatants were collected and used to inoculate fresh HFF and at 20 hours post-infection, lysates were collected and subject to anti-IE72 Western blot analysis, each point represents the average change in IE72 expression from two separate wells and error bars represent standard error; Rsqr=0.85 (A2t), 0.96 (p11);p<0.05;

FIG. 3B is a graphic showing the effect of purified annexin 2 on productive CMV infection in HFF in which HFF were inoculated with CMV at an MOI of 0.0003 in the presence of the indicated concentrations of purified AnxA2t (∘), p11 (●), or p36 ( [symbol] ), plaques were quantified on day 7 post-infection and each point represents the average change in plaque number from two separate wells, error bars represent standard error; Rsqr=0.95 (A2t), 0.95 (p11); p<0.05;

FIG. 4A is a graphic showing the effect of purified annexin 2 on early CMV infection stages in HFF in which HFF were inoculated with CMV at an MOI of 0.002 in the presence of the indicated concentrations of AnxA2t (∘), p11 (●), or p36 ( [symbol] ) and at 20 hours post-infection, lysates were collected and subject to anti-IE72 Western blot analysis, each point represents the average change in IE72 expression from two separate well and error bars represent standard error; Rsqr=0.91 (A2t), 0.94 (p11); p<0.05;

FIG. 4B is a graphic showing the effect of purified annexin 2 on productive CMV infection in HFF in which HFF were inoculated with CMV at an MOI of 0.0002 in the presence of the indicated concentrations of AnxA2t (∘), p11 (●), or p36 ( [symbol] ), plaques were quantified on day 7 postinfection and each point represents the average change in plaque number from two separate experiments, comprising two wells each, error bars represent standard error; p<0.05;

FIG. 5 is a graphic showing the effect of annexin 2 on early CMV infection stages in HepG2 where Native (∘) and p36-transfected (●) HepG2 cells were inoculated with CMV-infected cell supernatant at the indicated MOI and at 20 hours post-infection, lysates were collected and subject to anti-IE72 Western blot analysis, each point represents the average change in IE72 expression from two separate wells and error bars represent standard error; p<0.05, Rsqr=0.99 (for each);

FIG. 6 is an SDS-PAGE analysis of Cellular and viral experimental sources of AnxA1 and AnxA5 where total cell or purified CMV extracts (A), or supernatants of HFF, native HepG2 cells (nat) or p36-transfected HepG2 cells (p36) after 30 minutes at 37° C. in culture medium containing 1.8 mM calcium (B) or 20 mM EGTA (C), were subject to SDS-PAGE and immunoblot analysis using antibodies to AnxA5, AnxA1, p36, or NF-κB p65 subunit;

FIG. 7A is a graphic showing the effect of AnxA5 or AnxA1 on CMV infection of HFF in which HFF were inoculated with CMV at an MOI of 0.0003 in the presence of the indicated concentrations of AnxA5 (∘) or AnxA1 (●), on day 7 post-infection, supernatants were collected and used to inoculate fresh HFF and at 20 hours post-infection, cell lysates were collected and subject to anti-IE72 Western blot analysis, and each point corresponds to the average change from two separate experiments, comprising two three wells, error bars represent standard error; p<0.05;

FIG. 7B is a graphic showing the effect of AnxA5 or AnxA1 on CMV infection of HFF in which HFF were inoculated with CMV at an MOI of 0.0003 in the presence of the indicated concentrations of AnxA5 (∘) or AnxA1 (●). On day 7 post-infection, supernatants were collected and used to inoculate fresh HFF, plaques were quantified on day 6 post-infection, and each point corresponds to the average change from two separate experiments, comprising two three wells, error bars represent standard error; p<0.05;

FIG. 7C is a graphic showing the effect of AnxA5 or AnxA1 on CMV infection of HFF in which HFF were inoculated with CMV at an MOI of 0.003 in the presence of the indicated concentrations of AnxA5 (∘) or AnxA1 (●) and at 20 hours post-infection, cells lysates were collected and subject to anti-IE72 Western blot analysis, and each point corresponds to the average change from two separate experiments, comprising two three wells, error bars represent standard error; p<0.05;

FIG. 7D is a graphic showing the effect of AnxA5 or AnxA1 on CMV infection of HFF in which HFF were inoculated with CMV at an MOI of 0.0003 in the presence of the indicated concentrations of AnxA5 (∘) or AnxA1 (●), plaques were quantified on day 6 post-infection and each point corresponds to the average change from two separate experiments, comprising two three wells, error bars represent standard error; p<0.05;

FIG. 8A is a graphic showing the combined effect of AnxA5 or AnxA1 on primary plaque formation in HFF in which HFF were inoculated with CMV at an MOI of 0.0003 in the presence of the indicated concentration of AnxA1 alone (●) or with 50 nM AnxA5 (∘), plaques were quantified on day 6 post-infection, and each point corresponds to the average change in plaque number from two wells, error bars represent standard error; p<0.05;

FIG. 8B is a graphic showing the combined effect of AnxA5 or AnxA1 on primary plaque formation in HFF in which HFF were inoculated with CMV at an MOI of 0.0003 in the presence of the indicated concentration of AnxA5 alone (●) or with 5 nM AnxA1 (∘), plaques were quantified on day 6 post-infection and each point corresponds to the average change in plaque number from two wells, error bars represent standard error; p<0.05;

FIG. 9A is a graphic showing the effect of AnxA2 on AnxA5- or AnxA1-mediated attenuation of primary plaque formation in HFF in which HFF were inoculated with CMV at an MOI of 0.0003 in the presence of the indicated concentration of AnxA5 alone (∘) or pre-mixed with 25 nM AnxA2t (●), p11 ( [symbol] ), or p36 (▪), plaques were quantified on day 6 post-infection, and each point corresponds to the average change in plaque number from two wells, errorbars represent standard error; p<0.05;

FIG. 9B. is a graphic showing the effect of AnxA2 on AnxA5- or AnxA1-mediated attenuation of primary plaque formation in HFF in which HFF were inoculated with CMV at an MOI of 0.0003 in the presence of the indicated concentration of AnxA1 alone (∘) or premixed with 25 nM AnxA2t (●), p11 ( [symbol] ), or p36 (▪), plaques were quantified on day 6 postinfection and each point corresponds to the average change in plaque number from two wells, errorbars represent standard error; p<0.05;

FIG. 10A is a graphic showing the effect of inhibitory anti-AnxA2 antibodies on AnxA5- or AnxA1-mediated attenuation of primary plaque formation in HFF in which HFF were inoculated with CMV at an MOI of 0.0003 in the presence of the indicated concentration of AnxA5 alone (∘) or with 0.01 nM anti-p11 ( [symbol] ) or anti-p36 (▪) antibody, plaques were quantified on day 6 post-infection and each point corresponds to the average change in plaque number from two wells, error bars represent standard error; p<0.05;

FIG. 10B is a graphic showing the effect of inhibitory anti-AnxA2 antibodies on AnxA5- or AnxA1-mediated attenuation of primary plaque formation in HFF in which HFF were inoculated with CMV at an MOI of 0.0003 in the presence of the indicated concentration of AnxA1 alone (●) or with 0.01 nM anti-p11 ( [symbol] ) or anti-p36 (▪) antibody, plaques were quantified on day 6 post-infection and each point corresponds to the average change in plaque number from two wells, error bars represent standard error; p<0.05;

FIG. 11A is a graphic showing the effect of non-inhibitory anti-AnxA2 antibody (Translab anti-p36) on AnxA5- or AnxA1-mediated attenuation of primary plaque formation in HFF in which HFF were inoculated with CMV at an MOI of 0.0003 in the presence of the indicated concentration of anti-AnxA2 antibody alone ( [symbol] ) or with 250 nM AnxA5 (∘) or 100 nM AnxA1 (●) and each point corresponds to the average change in plaque number from two wells, error bars represent standard error; p<0.05;

FIG. 11B is a graphic showing the effect of non-inhibitory anti-AnxA2 antibody (Zymed anti-p36) on AnxA5- or AnxA1-mediated attenuation of primary plaque formation in HFF in which HFF were inoculated with CMV at an MOI of 0.0003 in the presence of the indicated concentration of anti-AnxA2 antibody alone ( [symbol] ) or with 250 nM AnxA5 (∘) or 100 nM AnxA1 (●) and each point corresponds to the average change in plaque number from two wells, error bars represent standard error; p<0.05;

FIG. 11C is a graphic showing the effect of non-inhibitory anti-AnxA2 antibody (Oncogene anti-p36) on AnxA5- or AnxA1-mediated attenuation of primary plaque formation in HFF in which HFF were inoculated with CMV at an MOI of 0.0003 in the presence of the indicated concentration of anti-AnxA2 antibody alone ( [symbol] ) or with 250 nM AnxA5 (∘) or 100 nM AnxA1 (●) and each point corresponds to the average change in plaque number from two wells, error bars represent standard error; p<0.05;

FIG. 11D is a graphic showing the effect of non-inhibitory anti-AnxA2 antibody (translab anti-p11) on AnxA5- or AnxA1-mediated attenuation of primary plaque formation in HFF in which HFF were inoculated with CMV at an MOI of 0.0003 in the presence of the indicated concentration of anti-AnxA2 antibody alone ( [symbol] ) or with 250 nM AnxA5 (∘) or 100 nM AnxA1 (●) and each point corresponds to the average change in plaque number from two wells, error bars represent standard error; p<0.05;

FIG. 12A is a graphic showing AnxA5 and AnxA1 in IE72 expression in native HepG2 cells in which HepG2 cells were inoculated at an MOI of 0.5 in the presence of the indicated concentrations of either AnxA5 (∘) or AnxA1 (●), at 20 hours post-infection, cells were lysed and subject to anti-IE72 Western blot analysis and each point corresponds to the average change in plaque number from two separate experiments comprising the average of two wells each, error bars represent standard error; p<0.05; and

FIG. 12B is a graphic showing AnxA5 and AnxA1 in IE72 expression in p-36 transfected HepG2 cells in which HepG2 cells were inoculated at an MOI of 0.5 in the presence of the indicated concentrations of either AnxA5 (∘) or AnxA1 (●), at 20 hours post-infection, cells were lysed and subject to anti-IE72 Western blot analysis and each point corresponds to the average change in plaque number from two separate experiments comprising the average of two wells each, error bars represent standard error; p<0.05.

It will be noted that throughout the appended drawings, like features are identified by like reference numerals.

### DETAILED DESCRIPTION OF THE PREFERRED EMBODIMENT

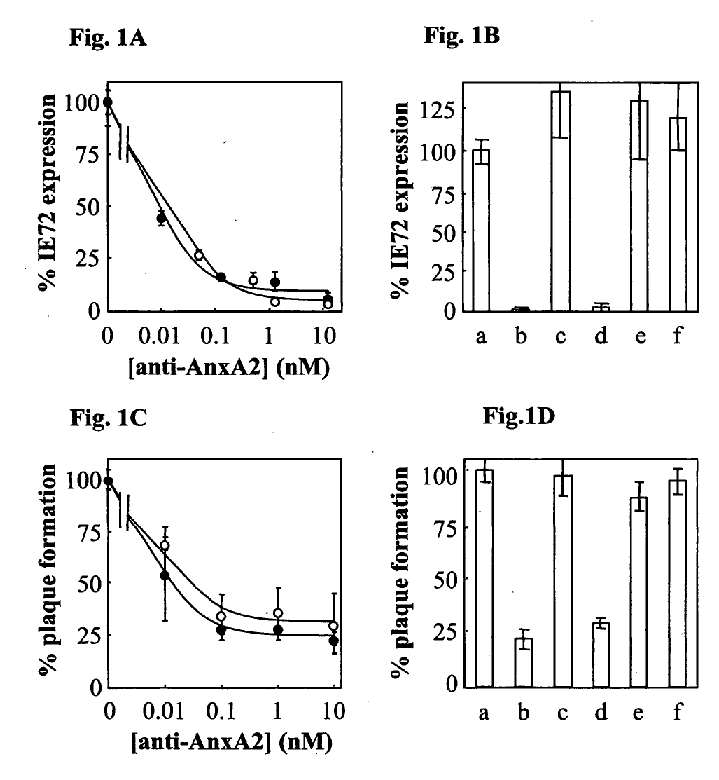

In one aspect, the present invention provides a method for inhibiting, preventing or otherwise reducing viral infections mediated by annexins. The method is useful for the treatment or prevention of viral infections in animals, including humans, that are infected by a virus or that are at risk of being infected. Effect of endogenous AnxA2 on CMV production of HFF. To determine the overall effect of AnxA2 in the multi-step infection process, the involvement of endogenous AnxA2 on the generation of CMV progeny was evaluated. As the first viral gene product, immediate-early 72 (IE72) generation, which has been previously used to assess penetration of CMV into the host cell (Compton et al., 1993, Virology 193, 834-841), was followed. Supernatants from HFF infected in the presence of either anti-p11 or anti-p36 antibody were used to inoculate fresh HFF in a second round of infection (i.e. two-step infection), and IE72 antigen was quantified by immunoblot analysis. Both anti-p11 (directed against p11 residues 21-38) and anti-p36 antibodies (directed against p36 residues 9-30) inhibited two-step IE72 expression by approximately 95% (FIG. 1A). Non-immune rabbit IgG, as well as anti-p11 and anti-p36 that had been pre-adsorbed with AnxA2t, had no significant effect on IE72 expression (FIG. 1B), indicating that the inhibition by anti-p11 and anti-p36 antibodies was specific. To confirm the two-step IE72 expression assays, two-step plaque assays were conducted to determine the effect of AnxA2 on the complete infection cycle. When supernatants from cells infected in the presence of anti-p11 or anti-p36 antibody were used to inoculate fresh HFF, second-generation CMV plaque formation was found to be inhibited in a dose-dependent manner to a maximum of approximately 70% (FIG. 1C). Neither non-immune rabbit IgG nor AnxA2t adsorbed anti-p11 or anti-p36 had a significant effect on plaque generation (FIG. 1D), demonstrating the antigenic specificity of inhibition. These results corroborate the two-step IE72 conclusions and suggest that endogenous p11 and p36, presumably associated as AnxA2t, enhance productive cell infection by CMV.

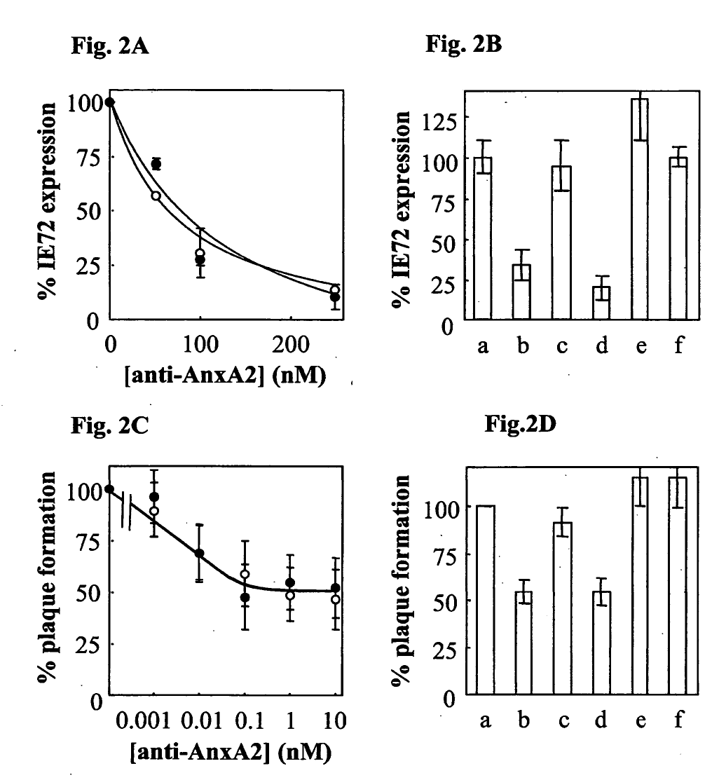

Effect of endogenous AnxA2 on primary CMV infection of HFF. The preceding two-step infection assays measured the combined effects of anti-p36 or anti-p11 on CMV entry, replication, egress and progeny viability. To narrow the focus of the investigation on steps preceding production of progeny virus, IE72 expression and plaque formation following the initial inoculation were investigated. Anti-p11 or anti-p36 antibody was observed to inhibit approximately 90% of primary (one-step) IE72 expression (FIG. 2A). Demonstrating specificity, neither non-immune rabbit IgG nor anti-p11 or anti-p36 antibody that had been preincubated with AnxA2t had a significant effect on IE72 expression (FIG. 2B). Other antibodies tested, including a mouse monoclonal anti-p11 antibody (Translab) and three mouse monoclonal anti-p36 antibodies (Translab, Zymed, or Calbiochem) had no significant effect on one-step IE72 expression (data not shown), suggesting that discrete epitopes of endogenous p36 or p11 enhance CMV infection at or prior to IE72 expression. To further investigate the effects of anti-p11 or anti-p36 antibody, the formation of primary (i.e. one-step) plaques was followed. This assay is typically used as a measurement of virus-cell entry but includes subsequent steps that are obligate for virus assembly and cytopathic effects (Greengard et al., 2000, J Viro 74, 11108-11114). Primary plaque number was reduced by 50% by anti-p11 or anti-p36 antibody (FIG. 2C). Non-immune rabbit IgG had no effect on plaque number, nor did anti-p11 or anti-p36 antibody that had been pre-incubated with AnxA2t (FIG. 2D), showing that inhibition by these antibodies was specific. Similar to primary IE72 expression, three other monoclonal anti-p36 antibodies and one other monoclonal anti-p11 antibody had no significant effect on primary plaque formation (data not shown). These data further show that endogenous p11 and p36 play a specific role to enhance CMV infection.

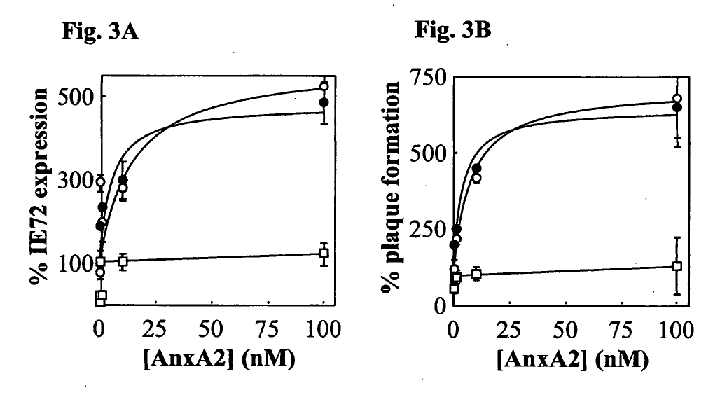

Effect of purified AnxA2 on CMV infection of HFF. To confirm the immunoinhibition of endogenous AnxA2 in CMV infection, and to determine whether the monomeric or tetrameric forms of AnxA2 are involved, the effect of purified AnxA2t, p11 or p36 was explored. The premise of these experiments was that the amount of endogenous AnxA2 was insufficient to maximize effects on CMV infection and that addition of purified AnxA2 mimicked the reported cell-surface induction of AnxA2 (Jacovina et al., 2001, J Biol Chem 276, 49350-49358; Peterson et al., 2003, J Cell Sci 116, 2399-2408). Supporting the immuno-inhibition experiments, either purified AnxA2t or p11, added only during the initial inoculation, enhanced two-step IE72 expression by progeny approximately 5-fold (FIG. 3A). In contrast, p36 had an insignificant effect on two-step IE72 expression, although dose-dependent binding of 125]-p36 to HFF was observed in separate experiments (data not shown), confirming a previously published report (Hajjar et al., 1996, J Biol Chem 271, 21652-21659). The effect of purified proteins on two-step IE72 expression was corroborated by plaque formation of CMV released into the media of infected cells. Purified AnxA2t and p11 were both observed to enhance the number of two-step plaques by approximately 7-fold (FIG. 3B). Again, added p36 had no significant effect, demonstrating the specificity of the enhancement by AnxA2t and p11.

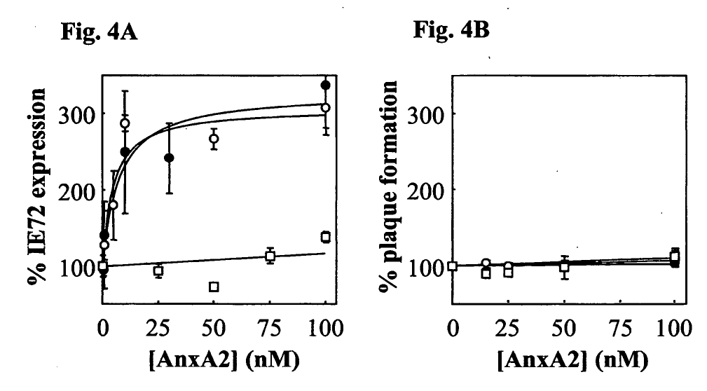

Effect of purified AnxA2 on primary CMV infection of HFF. To eliminate potential effects on egress and progeny viability inherent to our 2-step infection assays, IE72 expression and plaque formation following the initial inoculation were evaluated. Purified AnxA2t and p11 were found to enhance one-step IE72 expression by approximately 3-fold (FIG. 4A). As before, p36 had no significant effect. However, in contrast to our immunoinhibition experiments, which were consistent with a role for AnxA2 at this stage of infection (FIG. 2B), purified AnxA2t, p11, or p36 had no significant effect on one-step plaque number (FIG. 4B). These data highlight an interesting functional difference between purified and endogenous AnxA2 in CMV infection.

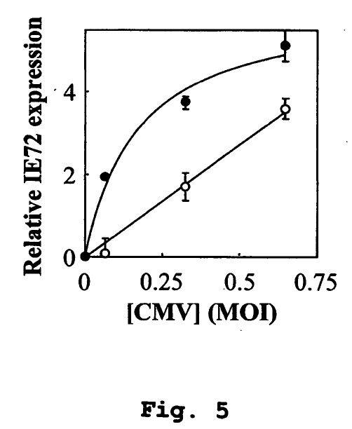

Effect of AnxA2 on early CMV gene expression in HepG2. To further support the role of AnxA2 observed for CMV infection of HFF, an additional cell model was employed. HepG2 cells are the only documented cell line that does not express either p36 mRNA or p36 antigen. While low levels of p11 mRNA and protein are detectable, transfection of HepG2 with the p36 gene (Puisieux et al., 1996, Biochem J 313 (PT 1), 51-55) increases p11 protein, suggesting formation of AnxA2t. Although neither native nor p36-transfected HepG2 cells facilitated productive CMV infection (data not shown), both were permissive to CMV entry, facilitating IE72 expression. The p36-transfected HepG2 cell line is therefore a useful tool for assessing the role of AnxA2 at early steps in CMV infection. To determine whether expression of p36 protein by HepG2 cells enhances early events in CMV infection, one-step IE72 assays were conducted. Detection of IE72 antigen was observed to be increased up to 3-fold in p36-transfected HepG2 cells, compared to native HepG2 (FIG. 5), indicating that endogenous AnxA2 enhances an early step in the CMV infection mechanism. These data furthermore confirm the results obtained with HFF and suggest that the cellular pool of AnxA2, and not just that endogenous to the virus surface, enhances CMV infection.

The effects of AnxA2 on CMV infection are summarized in table 1.

TABLE 1
Effect of annexin 2 on CMV infection*

Infection stage

| | IE72 | Plaques | IE72 | Plaques |
|---|---|---|---|---|
| Treatment | (2-step)^{†} | (2-step) | (1-step)^{‡} | (1-step) |
| Anti-p11 | 5 ± 3 | 30 ± 14 | 14 ± 4 | 47 ± 14 |
| Anti-p36 | 3 ± 1 | 32 ± 3 | 11 ± 8 | 53 ± 16 |
| AnxA2t | 520 ± 30 | 680 ± 20 | 310 ± 30 | NE^{§} |
| p11 | 480 ± 50 | 650 ± 10 | 340 ± 60 | NE |
| p36 | NE | NE | NE | NE |

*Percent of untreated.
^{†}In 2-step experiments, HFF were inoculated in the presence of the indicated treatment. Supernatants of infected cells were subsequently evaluated for presence of infectious progeny.
^{‡}In 1-step experiments, HFF were inoculated in the presence of the indicated treatment, and the effect on primary infection events was evaluated.
^{§}No effect.

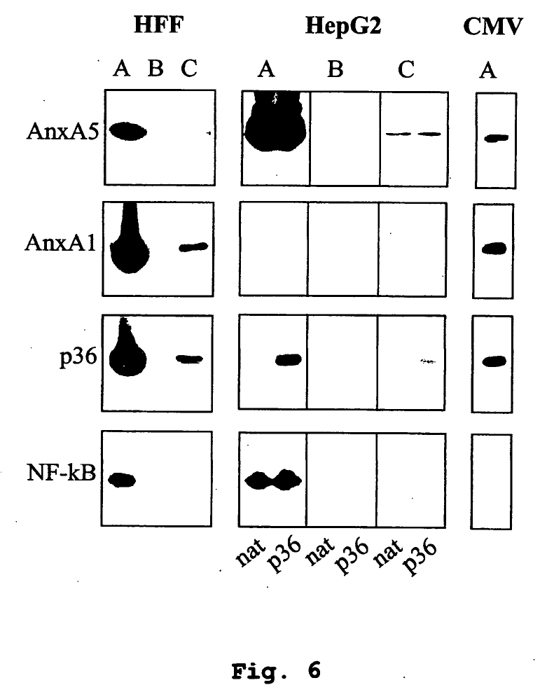

AnxA5 and AnxA1 expression on the surface of host cells. For AnxA5 and AnxA1 to participate during CMV infection, they must be available on the host cell surface to interact with other cellular and/or viral species during infection. Both AnxA1 (Castro-Caldas et al., 2002, Mol cell Biochem 237, 31-38; Chapman et al., 2003, Endocrinology 144, 1062-1073; Cover et al., 2002, Endocrine 18, 33-39; Solito et al., 2003, Endocrinology 144, 1164-1174) and AnxA5 (Krikun et al., 1994, Placenta 15, 601-612; Rand et al., 1994, Am J Obstet Gynecol 171, 1566-1572) have been reported on the surface of various cell types, but it is unknown whether they are available on the surface of cells to be used in the current study, HFF and HepG2. Since annexins interact with cell membranes in a calcium-dependent manner, the inventors determined whether AnxA5 or AnxA1 were detectable in the extracellular medium following EGTA elution from the cell surface, as previously described (Faure et al., 2002, Exp Cell Res 276, 79-89; Hajjar et al., 1994, J Biol Chem 269, 21191-21197), using Western blot analysis (FIG. 6).

For HFF, the typical laboratory model for CMV infection, AnxA5 and AnxA1 were detected in total cell lysates. Both annexins were also present in the EGTA eluate of non-lysed cells, but not in the control calcium eluate, indicating they interacted with the cell surface and required calcium to do so. A second cell model used in the current study is HepG2 cells, a cell line that does not express p36 (Puisieux et al., 1996, Biochem J 313 (PT 1), 51-55). Western blot analysis showed that AnxA5 was expressed in both cell lines and was also detected in the EGTA eluates, but not the calcium eluates. However, AnxA1 was not detected as a constituent of either native or p36-transfected HepG2 cells. As expected (Puisieux et al., 1996), p36 was detected in p36-transfected cells only, and was detected in the EGTA eluates, but not the calcium eluates. In addition to host cells, AnxA5 and AnxA1 were both detected in the lysate of purified virus preparations. As a positive control (Peterson et al., 2003, J Cell Sci 116, 2399-2408; Raynor et al., 1999, Biochemistry 38, 5089-5095), p36 was detected on the surface of HFF and in the CMV lysate. The intracellular protein NF-κB p65 subunit was not detectable in calcium or EGTA cell eluates, demonstrating that EGTA treatment enabled detection of species on the cell surface only. Collectively, these results demonstrate that AnxA5 and AnxA1 are available for interactions with other species on the cells and/or virus.

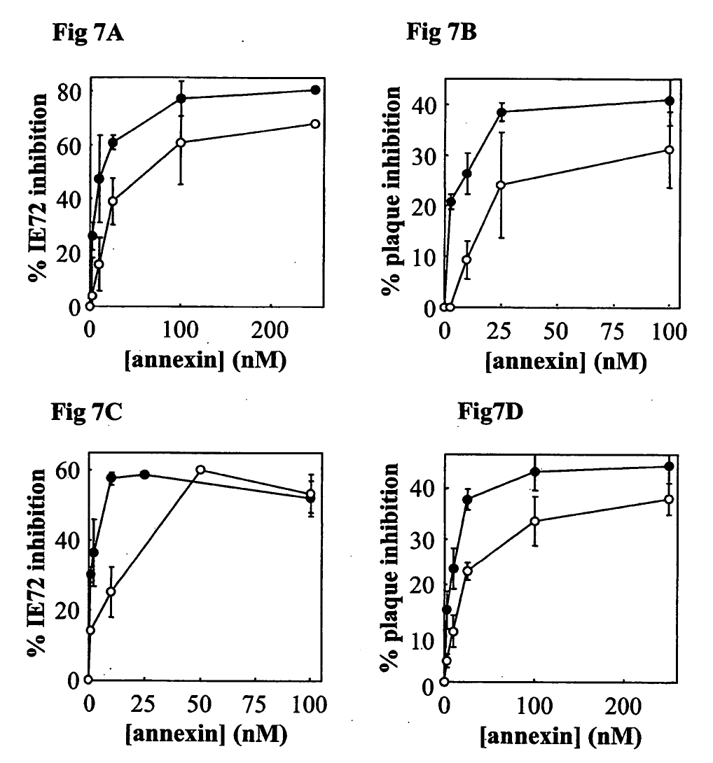

In another aspect of the invention, Annexin 5 (AnxA5) and annexin 1 (AnxA1) were shown to inhibit viral replication and infection. For example, AnxA5 and AnxA1 were shown to have an inhibitory effect on the overall Cytomegalovirus (CMV) replication mechanism, as measured by the generation of infectious progeny. Supernatants from Human Foreskin Fibroblast (HFF) infected in the presence of either AnxA5 or AnxA1 were used to inoculate fresh HFF in a second round of infection (i.e. two-step infection). CMV's Immediate Early 72 (IE72) antigen was subsequently quantified by immunoblot analysis to investigate effects on CMV generation. AnxA5 and AnxA1 each significantly inhibited two-step IE72 expression by approximately 80% (FIG. 7A). To confirm the two-step IE72 antigen assays, two-step plaque assays were conducted to determine the effect of AnxA5 or AnxA1 on the complete infection cycle. Similarly, second-generation CMV plaque formation was significantly inhibited by a maximum of approximately 45% (FIG. 7B). These results demonstrate that AnxA5 and AnxA1 each can inhibit production of infectious viral progeny.

Because endogenous AnxA2 (AnxA2) acts at least in part to increase CMV infection at a stage as early as IE72 expression (see below), experiments were conducted to assess the effect of AnxA5 and AnxA1 prior to the production of virus progeny. Either AnxA5 or AnxA1 significantly inhibited approximately 60% of primary IE72 expression (FIG. 7C), and primary plaque number was inhibited by approximately 45% (FIG. 7D). Taken together, these results demonstrated that the addition of purified AnxA5 or AnxA1 each specifically inhibit CMV infection of host HFF at a stage preceding IE72 expression. In agreement with previous reports (Pietropaolo et al., 1999, J Gen Virol 80 (Pt 7), 1807-1816) the p36 subunit of AnxA2t had no significant effect on any stage of CMV infection (data not shown), demonstrating the specificity of the inhibition by AnxA1 and AnxA5.

Effect of combined AnxA5 and AnxA1 on CMV infection. A consistent difference between AnxA5- and AnxA1-mediated CMV inhibition was that the concentration of half maximal inhibition (IC50) was 5- to 10-fold lower for AnxA1 than for AnxA5 (table 2).

TABLE 2
Effects of annexins 5 and 1 on CMV infection

| | 1-step | | 1-step | | 2-step | | 2-step | |
|---|---|---|---|---|---|---|---|---|
| | plaques* | | IE72 | | plaques^{†} | | IE72 | |
| | Iₘₐₓ^{‡} | IC₅₀^{§} | Iₘₐₓ | IC₅₀ | Iₘₐₓ | IC₅₀ | Iₘₐₓ | IC₅₀ |
| AnxA5 | 45 | 24 | 64 | 11 | 44 | 39 | 77 | 29 |
| AnxA1 | 43 | 5.0 | 58 | 1.1 | 42 | 3.4 | 81 | 6.0 |

*In 1-step experiments, HFF were inoculated in the presence of the indicated treatment, and the effect on primary infection events was evaluated.
^{†}In 2-step experiments, HFF were inoculated in the presence of the indicated treatment. Supernatants of infected cells were subsequently evaluated for presence of infectious progeny.
^{‡}Maximum inhibition = 100 − percent of untreated control.
^{§}Concentration at half-maximal inhibition (nM).

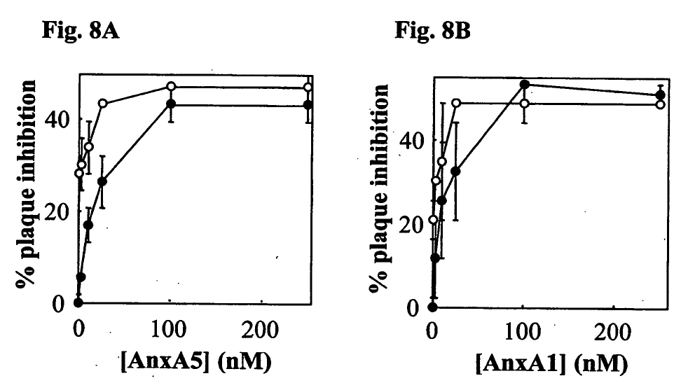

To investigate whether this observation is indicative of differing modes of inhibition, HFF were inoculated in the presence of either AnxA5 or AnxA1, or both, and primary plaque formation was quantified. In the presence of 5 nM AnxA1 (i.e. 50% of maximal inhibition alone), the apparent IC50 of AnxA5 was predictably decreased, due to an additive effect with AnxA1 (FIG. 8A). Similarly, in the presence of 50 nM AnxA5 (i.e. 50% of maximal inhibition alone), the apparent IC50 of AnxA1 was decreased due to an additive effect with AnxA5 (FIG. 8B). In both cases, the maximum inhibition observed (Imax) for AnxA5 and AnxA1 together was the same as for either alone (FIG. 8A, 8B). Therefore, these data suggest that AnxA5 and AnxA1 function independently, but affect the same saturable part of the CMV infection pathway.

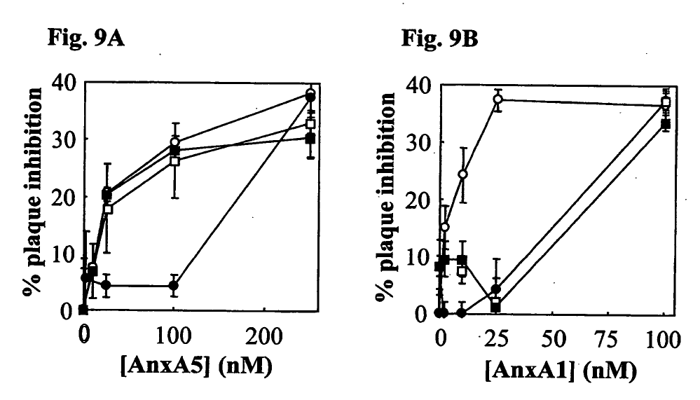

Effect of AnxA2 on AnxA5/1-mediated CMV attenuation. Because the preceding findings show that AnxA5 and AnxA1 inhibit the same steps of CMV infection shown previously to be enhanced by endogenous AnxA2t (supra), the inventors investigated whether AnxA5- and AnxA1-mediated inhibition of CMV infection was dependent on AnxA2. The first approach involved pre-incubating AnxA5 or AnxA1 with purified AnxA2 (25 nM) prior to inoculation. As shown previously (supra), and confirmed here, purified AnxA2t, p11, or p36 alone had no observable effect on primary plaque formation, thereby simplifying the interpretation of effects on AnxA5 or AnxA1. AnxA5-mediated inhibition of CMV infection was completely reversed by pre-incubation with purified AnxA2t, but was not significantly affected by the addition of purified p11 or p36 (FIG. 9A), demonstrating the specificity of the effect of AnxA2t on AnxA5. Restoration of expected inhibition was achieved in the presence of a 10-fold molar excess of AnxA5 (250 nM) over AnxA2t (25 nM), but not a 4-fold molar excess (100 nM). Similar to AnxA5, AnxA1-mediated inhibition could be completely reversed by pre-incubation with purified AnxA2t. However, unlike AnxA5, both p11 and p36 also reversed the inhibitory effect of AnxA1 (FIG. 9B) and a 4-fold molar excess of AnxA1 (100 nM) was sufficient to fully restore the expected extent of inhibition. These data suggest that inhibition of CMV infection by AnxA5 or AnxA1 is a consequence of reducing the enhancing effect of endogenous AnxA2.

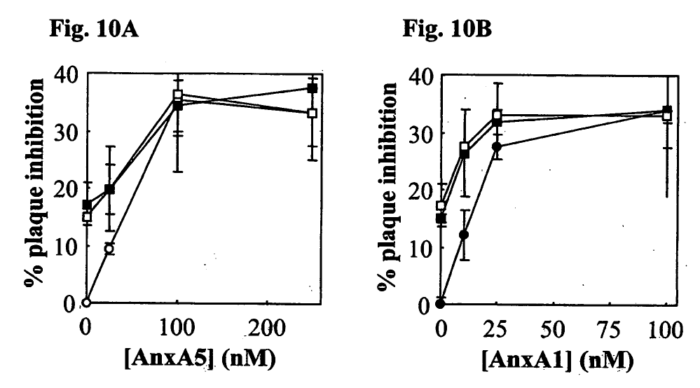

Effect of inhibitory anti-AnxA2 antibodies on AnxA5/1-mediated CMV attenuation. In a second approach taken to determine whether AnxA5- and AnxA1-mediated inhibition of CMV infection involves effects on endogenous AnxA2, the inventors employed anti-p11 and anti-p36 antibodies, previously shown to inhibit CMV plaque formation. The inventors hypothesized that the addition of AnxA5 or AnxA1 to these antibodies would not result in additional inhibition, since all are postulated to inhibit AnxA2-dependent CMV infection events. HFF were inoculated with CMV in the presence of anti-AnxA2 antibodies with either AnxA5 or AnxA1, and primary plaque formation was quantified. In the presence of 0.01 nM (i.e. nonsaturating) anti-p11 or anti-p36, the apparent IC50 for AnxA5 was observed at a lower concentration, consistent with an additive effect with each of the antibodies (FIG. 10A). Similarly, the apparent IC50 for AnxA1 was also observed at a lower concentration in the presence of anti-p11 or anti-p36, due to an additive effect (FIG. 10B). However, the addition of antibodies did not alter the Imax for either AnxA5 or AnxA1 (FIG. 5A, 5B). Non-immune rabbit IgG alone had no effect on infection, nor did it alter AnxA5 or AnxA1 activity (data not shown).

These observations suggest AnxA5, AnxA1, and anti-AnxA2 antibodies each target a similar CMV infection pathway for inhibition, further supporting the possibility that AnxA5 and AnxA1 inhibit infection by interfering with endogenous AnxA2-dependent events.

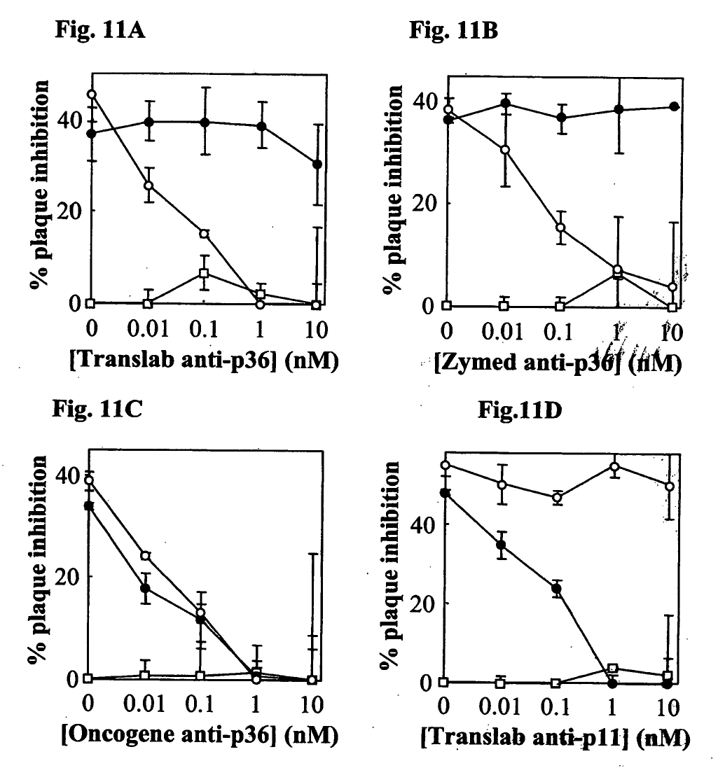

Effect of non-inhibitory anti-AnxA2 antibodies on AnxA5/1-mediated CMV attenuation. An additional approach taken to determine whether AnxA5- or AnxA1-mediated inhibition occurred by interfering with AnxA2 function employed anti-p11 and anti-p36 antibodies previously reported to have no significant effect on CMV infection (Pietropaolo et al., 1999, J Gen Virol 80 (Pt 7), 1807-1816). The inventors hypothesized that the addition of these antibodies may protect AnxA2 from the inhibitory effects of AnxA5 or AnxA1. HFF were inoculated with CMV in the presence of these “non-inhibitory” anti-AnxA2 mAbs with either AnxA5 (250 nM) or AnxA1 (100 nM), and primary plaque formation was quantified. As before, none of the antibodies alone had any significant effect on primary plaque formation (FIG. 6A-D). However, AnxA5-mediated inhibition was attenuated in a dose-dependent manner by Translab anti-p36 (FIG. 11A), Zymed anti-p36 (FIG. 11B) and Oncogene anti-p36 (FIG. 1C), but was unaffected by Translab anti-p11 (FIG. 11D). Interestingly, AnxA1-mediated inhibition was attenuated by a somewhat different set of anti AnxA2 antibodies: though neither Translab anti-p36 (FIG. 11A) nor Zymed anti-p36 (FIG. 11B) had any significant effect, both Oncogene anti-p36 (FIG. 11C) and Translab anti-p11 (FIG. 11D) reversed AnxA1-mediated inhibition. Non-immune mouse IgG isotype-matched controls had no significant effect alone or on inhibition by AnxA5 or AnxA1 (data not shown), indicating that the reversing effect of these anti-AnxA2 mAbs was specific. That the AnxA5- and AnxA1-mediated inhibition could be abrogated by the addition of select anti-AnxA2 antibodies, which alone had no effect on CMV plaque formation, suggests that AnxA5 and AnxA1 inhibition was dependent upon an interaction with AnxA2.

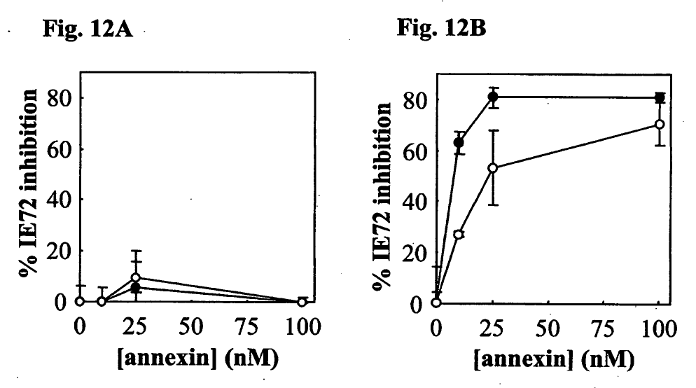

Effect of AnxA5/1 and AnxA1 on CMV infection of HepG2. To corroborate our HFF studies showing that AnxA5 or AnxA1 inhibit CMV infection, and to further investigate the possible AnxA2 dependence of this inhibition, a second cell model was used. Here the effect of AnxA5 and AnxA1 in the p36-null cell line, HepG2, was followed. HepG2 cells are the only known cell line in which neither p36 mRNA nor p36 protein can be detected (Puisieux et al., 1996, Biochem J 313 (Pt 1), 51-55), making them a useful model for studying AnxA2-related events in CMV infection by transfection. Although HepG2 cells are not permissive for productive CMV infection, transfection of p36 into HepG2 cells has been previously shown to increase primary IE72 expression by approximately 3-fold. To determine the influence of p36 expression on AnxA5- and AnxA1-mediated inhibition of CMV infection, HepG2 cells were inoculated in the presence of AnxA5 or AnxA1, and primary IE72 expression was quantified. In native HepG2 cells, neither AnxA5 nor AnxA1 had any significant effect on IE72 expression (FIG. 12A). However, in HepG2 that had been transfected with p36, both AnxA5 and AnxA1 significantly inhibited IE72 expression by approximately 80% (FIG. 12B). These observations further support the conclusion that AnxA5 and AnxA1 inhibit CMV infection by effects on AnxA2.

Cell culture assays such as plaque assays are well established assays to assess the efficacy of anti-viral agents and exhibit a high correlation with in vivo (animal or humans) efficacy. In this respect, animal models are well described in the prior art. Thus the antibodies and annexins that were shown, above, to be effective for preventing viral infections can be used in therapeutically effective amounts in animals in need thereof. By animal it is meant any animal, including humans, that is susceptible to viral infection the inhibition of which can be effected by the compounds mentioned above.

The term “therapeutically effective amount” as used herein for the treatment/reduction/prevention of viral infections refers to the amount of antibodies and/or annexins and anti-viral agents sufficient to reduce the number of viral particles or sufficient to prevent the initial infection and proliferation of viruses in an individual.

Determination of the need for treatment in individuals according to the present method in its various embodiments can be made by persons skilled in the art and may include, without being limited to, determination of viral load and polymerase chain reaction (PCR) techniques to assess the presence of viruses as well as epidemiologic data to assess the risk of infection in a given population.

The dosage ranges for the administration of the anti-viral compounds used in the present invention are those large enough to achieve the desired effect. The dosage may vary for example, with condition and age of the subject and the extent of infection and can be determined by those skilled in the art.

In particular the above mentioned compounds may be part of a pharmaceutical composition that can be administered intravenously, intramuscularly, subcutaneously, intraperitoneously or intraarterially or a combination thereof. The pharmaceutical composition may also comprise a pharmaceutically acceptable carrier. The administration of the pharmaceutical composition may also be topical to treat or prevent viral infections on the skin or mucosal surfaces for example. Pharmaceutically acceptable carrier for topical composition are well known in the art.

Thus Anti-p11 and anti-p36 antibodies, AnxA1, AnxA5 or a combination thereof can be administered to an animal in need thereof using method well known in the art. In addition, the above mentioned compounds can be used in combination with one or more known anti-viral agents to enhance the efficacy of the treatment. Anti-viral agents may comprise, but are not limited to, acyclovir, viroptic, idoxirudine, saquinavir, nevirapine and the like. Enhancement of the efficacy of anti-viral agents by antibodies are known in the art as described for example in Nokta et al. Antiviral Res. 1994, 24(11):17-26.

It will be appreciated that viral infection mediated by AnxA2 may also be inhibited or reduced by preventing the transcription and/or the translation of the gene coding for AnxA2. For example, anti-sense RNA therapy techniques are envisioned to be covered by the present invention.

While the method for inhibiting/reducing/preventing of the present invention has been demonstrated cell membrane-enveloped viruses such as the cytomegalovirus (CMV), it can be applied to any virus the infection cycle of which is dependent in part on AnxA2 and or AnxA1/5.

In yet another aspect of the invention, there is provided a method for assessing the susceptibility of an animal to viral infection comprising measuring the levels of AnxA1, AnxA2 and/or AnxA5 in a given tissue of an animal. Increased levels of AnxA2 indicating an increase susceptibility to viral infection and increased levels of AnxA1 and/or AnxA5 indicating a decrease susceptibility to viral infection. It will be appreciated that relative variations in the levels of annexins in a given tissue of an animal may be based on a comparison with an established standard for the tissue or with previous measurements made on the tissue from the same animal. It will also be appreciated that relative levels of AnxA2 vs. AnxA1 and/or AnxA5 may also be indicative of susceptibility to viral infection. More specifically, increased ratios of AnxA2 vs AnxA1 and/or AnxA5 indicating a higher susceptibility and a decrease in that ratio indicating a lower susceptibility.

By tissue it is meant any animal tissue, including blood, that is amenable to annexins measurement.

It is also possible to take advantage of the properties of AnxA1, AnxA2 and AnxA5 with regard to viral infection for increasing the efficiency of viral entry into cells for the purpose of therapy such as gene therapy mediated by viral vectors. Thus, for example, exogenous AnxA2 can be added to cells targeted for therapy thereby increasing viral entry into such cells. A similar effect can be achieved by reducing the inhibitory effect of AnxA1 and AnxA5.

Modulation of viral infection efficiency can also be achieved by modifying the relative amount of annexin types at the surface of the cells using therapeutics such as annexin analogs.

### EXAMPLES

Materials and Methods

Materials. Human foreskin fibroblasts (HFF) were obtained from American Tissue Culture Collection (ATCC, CRL-2056), and were grown and maintained in Basal Medium Eagle containing 5% bovine calf serum, 14 μM L-glutamine, and 1 U ml-1 gentamycin (BME). Native HepG2 cells were obtained from ATCC (HB-8065), and were grown and maintained in Minimum Essential Medium containing 10% fetal calf serum, 14 μM L-glutamine, and 1 U ml-1 gentamycin (MEM). HepG2 cells transfected with p36 were a kind gift from Dr. Alain Puisieux (Centre Anti cancer Leon-Berard, Lyon, France), and were grown and maintained in MEM containing 0.4 mg ml-1 geneticin. CMV (AD-169 strain) was propagated in HFF, purified by tartrate glycerol ultracentrifugation, and quantified by electron microscopy and plaque assay as previously described (Pryzdial et al., 1994, Blood 84, 3749-3757). Purified AnxA2t and p11, derived from bovine lung, and recombinant human p36 were prepared and characterized as previously described (Kang et al., 1997, Biochemistry 36, 2041-2050; Khanna et al., 1990, Biochemistry 29, 4852-4862). Purified recombinant AnxA5 was obtained from Pharmingen. Purified AnxA1 was prepared from human placenta as previously described for AnxA2 (Raynor et al., 1999), with an additional purification step using an anti-AnxA1 immuno-affinity column from which the AnxA1 was eluted using 3M KSCN, then changed into HBS using 10-kDa cutoff Centricon filters. Rabbit polyclonal anti-p11 antibody, directed against p11 residues 21-38, was generated and purified as previously described (Kassam et al., 1998, Biochemistry 37, 16958-16966; Peterson et al., 2003).

Rabbit polyclonal anti-p36 antibody, specific to a peptide corresponding to residues 9-30, was obtained from BioDesign. Mouse monoclonal antibodies (mAbs) directed against the following antigens were obtained commercially: AnxA1 (Translab), p36 (Translab, Oncogene, or Zymed as indicated), p11 (Translab), AnxA5 (Alexis), NF-κB p65 subunit (Translab), CMV immediateearly 72 (IE72) product (Accurate Scientific), and β-actin (Sigma-Aldrich).

Cell surface protein elution by EGTA. HFF or HepG2 cells, grown to confluence, were washed three times and incubated in serum-free BME in the presence or absence of 20 mM EGTA for 30 minutes at 37° C. Supernatants were clarified at 2500 g for 5 minutes and diluted in SDS-PAGE sample buffer (2% SDS, 0.5 M Tris, 10% glycerol, 12.5 mg ml-1 dithiothreitol). Cell lysates were obtained by washing confluent cells in serum-free BME, then dissolving in SDS-PAGE sample buffer. All samples were subsequently boiled for 10 minutes, resolved by SDS-PAGE, and subject to Western blot analysis using mAbs directed against AnxA1, AnxA5, p36, or NF-κB p65 subunit. Inoculations. In all infection assays, HFF were grown to approximately 80% confluence. Antibody and purified protein were pre-incubated on ice for 60 minutes prior to incubating with cells and virus. In antibody inhibition assays, cells were pre-washed twice in serum-free Basal Medium Eagle supplemented with 1 mg ml-1 bovine serum albumin (BME/BSA) containing 20 mM EGTA (Hajjar et al., 1996, J Biol Cheml 271, 21652-21659)), and washed twice in BME/BSA alone. In purified protein assays, cells were pre-washed twice in BME/BSA containing 2 mM CaCl₂ (BME/BSA/Ca). Both cells and virus were separately pre-incubated, at 37° C. and on ice respectively, with antibody or protein for 60 minutes. Cells were inoculated with CMV (final volume of 200 μl) at the indicated multiplicity of infection (MOI), and incubated for 90 minutes at 37° C. Cells were then washed three times in BME and cultured in BME to allow infection to develop. In HepG2 experiments, inoculations were performed in Minimum Essential Medium containing 1 mg ml-1 BSA and 2 mM CaCl₂ (MEM/BSA/Ca), using CMV-infected HFF supernatant (Pryzdial et al., 1994, Blood 84, 3749-3757) for inoculum, at the indicated MOI.

Two-step IE72 assays. Infectious progeny release from host cells was measured by assessing infected cell supernatants for the ability to generate the IE72 gene product. Following inoculation, infection was allowed to develop for 10 days, with medium replacement on days 3 and 5 post-infection. On day 10 post-infection, supernatants from infected cells were collected and used to inoculate fresh HFF monolayers. At 20 hours post-infection, the cells were lysed in SDS-PAGE sample buffer. Samples were then boiled for 10 minutes, resolved by SDS-PAGE, and subject to anti-IE72 Western blot analysis.

Two-step plaque assays. Infectious progeny release was measured by plaque assay of infected cell supernatants. Following inoculation, infection was allowed to develop for 10 days, with medium replacement on days 3 and 5 post-infection. On day 10 post-infection, supernatants from infected cells were collected and used to inoculate fresh HFF monolayers for 90 minutes at 37° C. This second round of infection was allowed to develop for 7 days, with medium replacement on days 3 and 5 post-infection. Plaques were counted on day 7 post-infection.

One-step IE72 assays. Early post-entry infection events were evaluated by IE72 expression shortly after primary inoculation. Since IE72 expression and plaque formation are logarithmically proportional (data not shown), MOI was therefore selected to ensure that the amount of virus was proportional to IE72 expression while still permitting early detection of infectious events. Following inoculation, infection was allowed to develop for 20 hours. The cells were then lysed in SDS-PAGE sample buffer, boiled for 10 minutes, resolved by SDSPAGE, and subject to anti-IE72 Western blot analysis.

One-step plaque assays. Early infection events leading up to and including CMV cell entry were measured by changes in plaque number (Greengard et al., 2000, J Virol 74, 11108-11114). Following inoculation, infection was allowed to develop for 7 days, with medium replacement on days 3 and 5 post-infection. Plaques were counted on day 7 post-infection.

Western blot. Detection of antigen by Western blot was performed according to the enhanced chemiluminescence method (Amersham-Pharmacia). Briefly, polyvinyl difluoride (PVDF) membranes were blocked in 5% skim milk powder in Tris-buffered saline (0.5 M Tris, 150 mM NaCl, pH 8) containing 0.05% (w/v) Tween-20 (TBST) for 60 minutes. Membranes were incubated with anti-IE72 antibody in 5% skim milk in TBST for 60 minutes, washed in TBST, incubated with secondary horseradish peroxidase-conjugated goat anti-mouse antibody (Jackson) in TBST for 30 minutes, and washed again in TBST. Western blots were developed as per the manufacturer's instructions. Blots were subsequently reprobed for β-actin. IE72 band intensity, relativized to β-actin band intensity, was quantified using Northern Eclipse imaging software (Empix).

Data analysis. Percentage of inhibition of infection was determined using the following formula: % inhibition=100−(% infection of the control). To calculate maximum inhibition (Imax) and concentration required to reach half-maximum inhibition (IC50), all titration data were fit by non-linear least squares to a simple mathematical model that assumed a single class of binding for either antibody or purified AnxA2 (i.e. a rectangular hyperbola), using SigmaPlot graphing software (Jandel Scientific). Statistical significance was further analyzed using a two-tailed T-test (>95% confidence), by comparing individual points to the control.

The embodiment(s) of the invention described above is(are) intended to be exemplary only. The scope of the invention is therefore intended to be limited solely by the scope of the appended claims.

I/We claim:

1. A method for preventing or treating a viral infection in an animal in need thereof comprising administering a therapeutically effective amount of an inhibitor of heterotetrameric annexin 2.

2. The method as claimed in claim 1, wherein said inhibitor is selected from an anti-heterotetrameric annexin 2 antibody, annexin 1, annexin 5 and a combination thereof.

3. The method as claimed in claim 1, wherein said viral infection is caused by a cell-membrane enveloped virus.

4. The method as claimed in claim 3, wherein said cell-membrane enveloped virus is cytomegalovirus (CMV).

5. The method as claimed in claim 1, wherein said heterotetrameric annexin 2 inhibitor is administered in combination with at least one anti-viral agent.

6. The method as claimed in claim 1 wherein said animal is a human.

7. The method as claimed in claim 6, wherein said heterotetrameric annexin 2 inhibitor is administered as a pharmaceutical composition further comprising a pharmaceutically acceptable carrier.

8. The method as claimed in claim 7, wherein said pharmaceutical composition is administered intravenously, intramuscularly, subcutaneously, intraperitoneously, intraarterially, topically or a combination thereof.

9. A method for preventing or treating a viral infection in an animal in need thereof comprising administering a therapeutically effective amount of annexin 1 and/or annexin 5 or an analog thereof to said animal.

10. The method as claimed in claim 9, wherein said viral infection is caused by a cell-membrane enveloped virus.

11. The method as claimed in claim 10, wherein said cell-membrane envelop virus is cytomegalovirus (CMV).

12. The method as claimed in claim 9, wherein said annexin 1 and/or annexin 5 or said analog thereof is administered in combination with at least one anti-viral agent.

13. The method as claimed in claim 9, wherein said animal is a human.

14. The method as claimed in claim 13, wherein said annexin 1 and/or annexin 5 or said analog thereof is administered as a pharmaceutical composition further comprising a pharmaceutically acceptable carrier.

15. The method as claimed in claim 14, wherein said pharmaceutical composition is administered intravenously, intramuscularly, subcutaneously, intraperitoneously, intraarterially, topically or a combination thereof.

16. A method for assessing susceptibility of an animal to viral infection, said method comprising providing a tissue sample from said animal and measuring levels of annexin 1 and/or annexin 5 in said tissue to determine whether said levels are increased or decreased relative to a predetermined standard level, said increased being an indication of lower susceptibility and said decrease being an indication of higher susceptibility.

17. The method as claimed in claim 16, wherein said levels are compared to levels of annexin 2 to generate a ratio of annexin 1 and/or annexin 5 to annexin 2 and wherein said ratio is compared to a predetermined ratio, said predetermined ratio being indicative of a degree of susceptibility and wherein ratios higher than said predetermined ratio being an indication of lower susceptibility and ratios lower than said predetermined ratio being an indication of higher susceptibility.

18. The method as claimed in claim 16, wherein said levels of annexin 1 and/or 5 are considered together with levels of a marker of viral infection susceptibility other than annexins.

19. The method as claimed in claim 18, wherein said marker is selected from heparan sulfate proteoglycan, epidermal growth factor receptor and CD13.

20. A method for assessing susceptibility of an animal to viral infection, said method comprising providing a tissue sample from said animal and measuring levels of annexin 2 in said tissue to determine whether said levels are increased or decreased relative to a predetermined standard level, said increased being an indication of higher susceptibility and said decrease being an indication of lower susceptibility.

21. The method as claimed in claim 20, wherein said levels of annexin 2 are considered together with levels of a marker of viral infection susceptibility other than annexins.

22. The method as claimed in claim 21, wherein said marker is selected from heparan sulfate proteoglycan, epidermal growth factor receptor and CD13.

23. A method for increasing viral infection of cells comprising contacting said cells with exogeneous heterotetrameric form of annexin 2.

24. The method as claimed in claim 23, wherein said increase in viral infection is used to increase efficiency of gene therapy mediated by virus vectors.

25. A method for increasing viral infection of cells comprising attenuating an inhibitory effect of annexin 1 and/or annexin 5.

26. The method as claimed in claim 25, wherein said increase in viral infection is used to increase efficiency of gene therapy mediated by virus vectors.

27. A method for modulating viral infection in cells comprising treating said cells with an agent capable of modifying a ratio of annexin types on the surface of said cells.

28. The method as claimed in claim 27, wherein said agent is an annexin analogue.
